(19) United States
(12) Patent Application Publication
(10) Pub. No.: US 2015/0088229 A1
(43) Pub. Date: Mar. 26, 2015

(54) Method and System to Remotely Control a Transcutaneous Electrical Nerve Stimulation Device

(71) Applicant: Zack Benbassat, Orlando, FL (US)

(72) Inventor: Zack Benbassat, Orlando, FL (US)

(21) Appl. No.: 14/032,698

(22) Filed: Sep. 20, 2013

(51) Int. Cl. A61N 1/36

(52) U.S. Cl. CPC A61N 1/36014
USPC 607/60

(57) ABSTRACT

A system to remotely control a transcutaneous electrical nerve stimulation (TENS) device is disclosed. The system includes a TENS control unit where a transceiver of the TENS control unit is configured to receive wireless Bluetooth signals transmitted from a smartphone, and to transmit wireless Bluetooth response signals to the smartphone in response to biofeedback from a user's body. The system also includes a controller configured to transmit electrical stimulus pulses during a treatment session in response to receiving the wireless Bluetooth signals, where the controller is configured to modulate pulse width, frequency, intensity, or any combination thereof, of the electrical stimulus pulses. A graphical user interface is accessible using the smartphone and configured for a user to select desired control commands to transmit to the TENS control unit using the wireless Bluetooth signals, where the control commands determine the electrical stimulus impulses that are applied to the user's body.

### I. FIELD

The present invention relates in general to a method and system to remotely control a transcutaneous electrical nerve stimulation device.

### II. DESCRIPTION OF RELATED ART

Transcutaneous electrical nerve stimulation (TENS) has been an accepted mode of electrotherapy for many years. TENS is primarily intended for pain relief via a nerve signal blocking mechanism, but it has also been used to promote healing. TENS devices typically deliver biphasic stimulus 30 pulses between 10 milliamperes (mA) and 100 mA in amplitude. The stimulus parameters that define the stimulus treatment include a pulse amplitude, pulse width and pulse rate selected by a user. The stimulus is delivered to a pair of electrode pads that are strategically placed over major muscle groups or nerves that are to receive the stimulation.

Electrode pad placement and the manner of stimulation is critical to effective treatment. However, patients often lack the knowledge needed to effectively place the electrode pads by themselves or to select the appropriate stimulation protocol, which necessitates frequent physician office visits. Thus, there is a need for a TENS device that can be automatically and remotely configured to deliver the most recent and appropriate stimulation protocol for a variety of different treatment conditions depending on the needs of the patient without physician supervision.

However, in view of the prior art at the time the present invention was made, it was not obvious to those of ordinary skill in the pertinent art how the identified needs could be fulfilled.

### III. SUMMARY

In a particular embodiment, a system to remotely control a transcutaneous electrical nerve stimulation device is disclosed. The system includes a TENS control unit, a power source within the TENS control unit, a transceiver of the TENS control unit is configured to receive wireless Bluetooth signals transmitted from a smartphone, and a controller of the TENS unit is configured to transmit electrical stimulus pulses during a treatment session in response to receiving the wireless Bluetooth signals. In addition, the system includes electrode pads in electrical communication with the TENS control unit and configured to apply the electrical stimulus pulses to a user's body. A graphical user interface (GUI) may be accessible using the smartphone and be configured for a user to select desired control commands to transmit to the TENS control unit using the wireless Bluetooth signals, where the control commands determine the electrical stimulus impulses that are applied to the user's body. The GUI may be installed on the smartphone or accessible over an intranet or Internet using the smartphone. The controller is configured to modulate pulse width, frequency, intensity, or any combination thereof, of the electrical stimulus pulses.

Further, the GUI may be configured for a user to select a duration of the electrical stimulus pulses. The transceiver of the TENS control unit may also be configured to transmit wireless Bluetooth response signals to the smartphone in response to biofeedback from the user's body, where the GUI is configured to automatically adjust the control commands to transmit to the TENS control unit in response to receiving the response signals of the biofeedback. The GUI may also be configured to store the biofeedback and the control commands to access in a subsequent treatment session.

In another particular embodiment, a method to remotely control a transcutaneous electrical nerve stimulation (TENS) device is disclosed. The method includes transmitting wireless Bluetooth signals to a TENS control unit from a smartphone, receiving the wireless Bluetooth signals at a transceiver of the TENS control unit, and transmitting electrical stimulus pulses from the TENS control unit for application to a user's body during a treatment session in response to receiving the wireless Bluetooth signals. The method also includes providing electrode pads in electrical communication with the TENS control unit to apply the electrical stimulus pulses to the user's body. In addition, the method may include accessing a graphical user interface using the smartphone, and selecting desired control commands to transmit to the TENS control unit using the wireless Bluetooth signals, where the control commands determine the electrical stimulus impulses that are applied to the user's body. The GUI may be installed on the smartphone, or accessible over an intranet or Internet using the smartphone. The method may include modulating a pulse width, frequency, intensity, or any combination thereof, of the electrical stimulus pulses, and selecting a duration of the electrical stimulus pulses. Further, the method may include transmitting wireless Bluetooth response signals to the smartphone in response to receiving biofeedback from the user's body, and automatically adjusting the control commands to transmit to the TENS control unit in response to receiving the response signals of the biofeedback.

Other aspects, advantages, and features of the present disclosure will become apparent after review of the entire application, including the following sections: Brief Description of the Drawings, Detailed Description, and the Claims.

### IV. BRIEF DESCRIPTION OF THE DRAWINGS

FIG. 1 is a schematic view of a particular illustrative embodiment of a system to remotely control a transcutaneous electrical nerve stimulation (TENS) device;

FIG. 2 is a block diagram of a particular illustrative embodiment of the system to remotely control the TENS device of FIG. 1; and

FIG. 3 is a flow diagram of a particular illustrative embodiment of a method to remotely control the TENS device.

### V. DETAILED DESCRIPTION

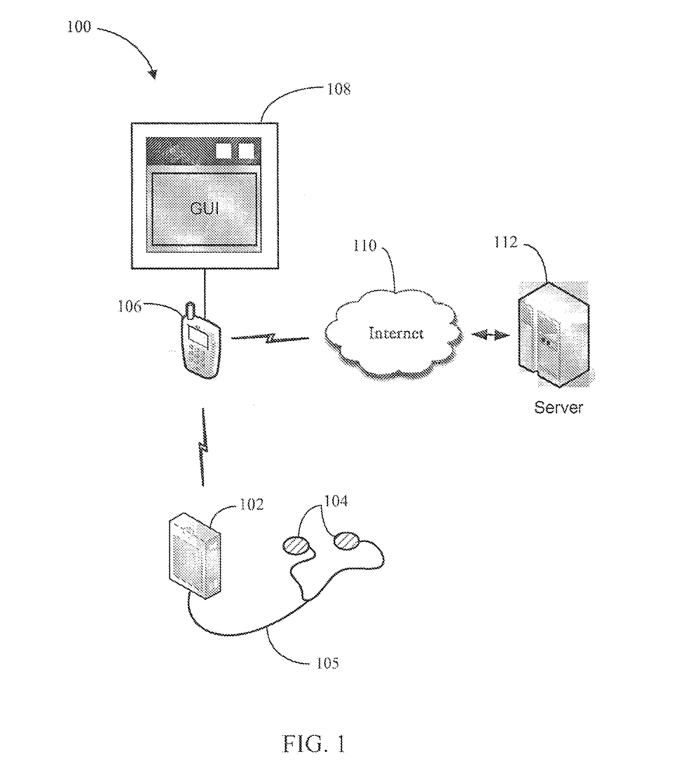

FIG. 1 is a schematic view of an illustrative system to remotely control a transcutaneous electrical nerve stimulation (TENS) device, generally designated 100. The system 100 includes a TENS control unit 102 and electrodes pads 104 connected to the TENS control unit 102 by a pair of lead wires 106. The electrode pads 104 placement over precise anatomical locations may be adjusted for use at anatomical sites of muscular-tendonous breakdown points surrounding the ankle, knee, hip, wrist, elbow, shoulder, neck and back. The TENS control unit 102 may include control buttons, a power supply, such as a battery, and a plurality of indicator lights that allow the user to verify the mode and intensity selected for the treatment protocol.

In operation, a smartphone 106 may be used to remotely control the TENS control unit 102 using Bluetooth wireless signals. A graphical user interface (GUI) 108 may be installed on the smartphone 106 or the GUI 108 may be accessible from a remote server 112 via the Internet 110, intranet, or other network. The GUI 108 may display a screen or drop down menu so that the user may select visually a body part that is intended to receive treatment, for example. Next, the GUI 108 provides at least one treatment protocol that is available to be transmitted to the TENS control unit 102. The user selects the desired treatment protocol using the GUI 108, which then transmits the treatment protocol to the TENS control unit 102 using wireless Bluetooth signals, for example. The TENS control unit 102 receives the signals transmitting the desired treatment protocol. An indicator light of the TENS control unit 102 may indicate that the treatment protocol has been received and is ready to deliver the stimulus pulses to the electrode pads 104 in accordance with the treatment protocol. The user toggles the TENS control unit 102 and the treatment is delivered to the user according to the selected treatment protocol.

A variety of information may also be displayed on the GUI 108. For example, the information may include a patient name and history of treatment. In addition, the information on the GUI 108 may include tabs that a user may toggle to access additional screens used to enter information to remotely configure the treatment protocol. A server 112 may be adapted to serve up the GUI 108 and other screens generated by the system 100. Additionally, the GUI 108 may be used to suggest a treatment protocol.

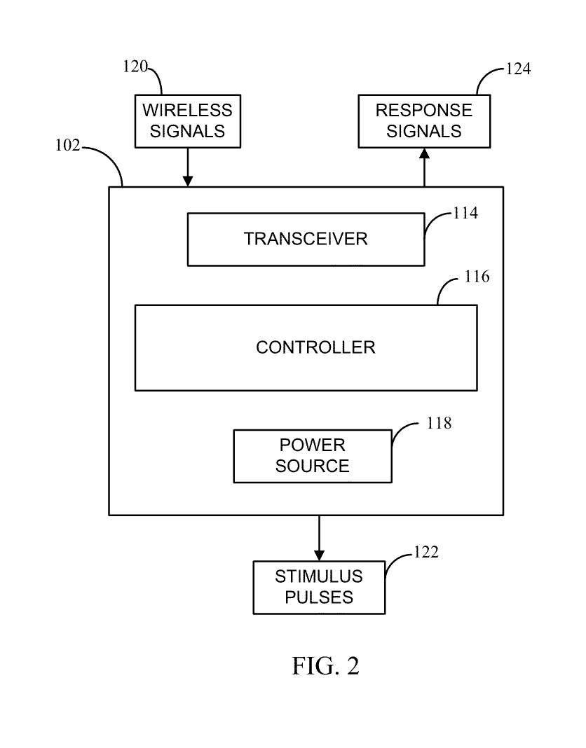

Referring now to FIG. 2, the TENS device 102 includes a controller 116 that regulates operation of the TENS unit 102 through a plurality of different treatment modes, each mode may be directed to treat a specific physical condition or anatomical location. This may be determined by the treatment protocol selected by the user. The controller 116 may be operated by a microcontroller integrated circuit or other means well known in the art. The controller 116 permits a manual selection of signal mode and selection of intensity by manipulation of the control buttons by the user or by a treatment protocol received via the smartphone 106 described above. A transceiver 114 of the TENS control unit 102 is configured to receive wireless signals 120, such as Bluetooth signals, from the smartphone 106. The transceiver 114 is in communication with the controller 116 which controls the delivery of the stimulus pulses 122 to the user.

The treatment protocol determines the parameters of the stimulus pulses 122, where the parameters may include a pulse amplitude, pulse width and pulse rate. The treatment protocol may be based on particular symptoms, anatomical location of treatment, biofeedback response signals 124 from the user, or any combination thereof. The response signals 124 may be based on the user's biofeedback from the stimulus pulses 122 and are transmitted by the transceiver 114 to the smartphone. The response signals 124 may be analyzed by the GUI to be able to modify the treatment protocol for a particular user and also the response signals 124 may be stored to create a history of the user's reaction to particular treatment protocol. Accordingly, the treatment protocol may be modified to generate another protocol that with the intent to improve the efficacy of treatment of the patient's ailments.

The system 100 may include a number of predetermined treatment protocols for selection by the user. At least some of the predetermined treatment protocol options may be presented in drop down menus. For example, the predetermined treatment protocols may include, but are not limited to, pulse amplitude, pulse width and pulse rate. In some cases, the TENS control unit 102 may download configuration information for the treatment protocols from the server 112.

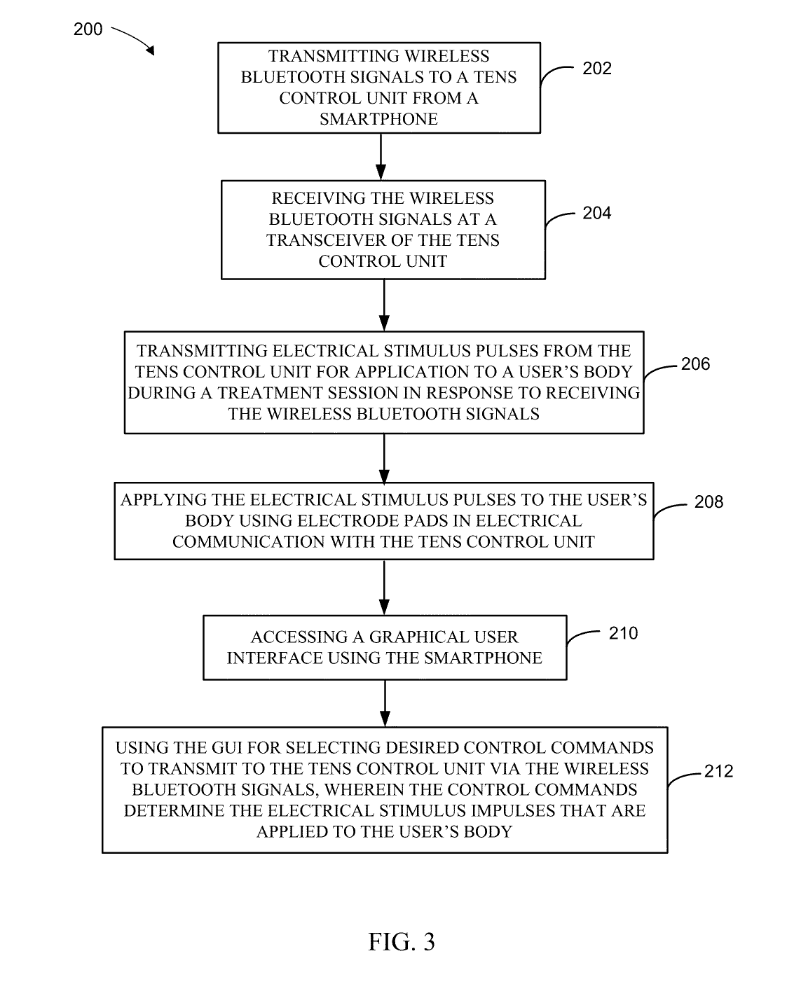

A flow diagram of a particular embodiment of a method to remotely control a TENS device is designated 200 and described in FIG. 3. At 202, wireless Bluetooth signals are transmitted to a TENS control unit from a smartphone. Moving to 204, the wireless Bluetooth signals are received at a transceiver of the TENS control unit. Electrical stimulus pulses are transmitted, at 206, from the TENS control unit for application to a user's body during a treatment session in response to receiving the wireless Bluetooth signals. At 208, the electrical stimulus pulses are applied to the user's body using electrode pads in electrical communication with the TENS control unit. A graphical user interface (GUI) is accessed using the smartphone, at 210. Desired control commands are selected, at 212, using the GUI to transmit to the TENS control unit via the wireless Bluetooth signals, wherein the control commands determine the electrical stimulus impulses that are applied to the user's body.

The previous description of the disclosed embodiments is provided to enable any person skilled in the art to make or use the disclosed embodiments. Various modifications to these embodiments will be readily apparent to those skilled in the art, and the principles defined herein may be applied to other embodiments without departing from the scope of the disclosure. Thus, the present disclosure is not intended to be limited to the embodiments shown herein but is to be accorded the widest scope possible consistent with the principles and novel features as defined by the following claims.

1. (canceled)

2. (canceled)

3. (canceled)

4. (canceled)

5. (canceled)

6. (canceled)

7. (canceled)

8. (canceled)

9. (canceled)

10. (canceled)

11. A method to remotely control a transcutaneous electrical nerve stimulation (TENS) device, the method comprising:
accessing a graphical user interface (GUI) using a smartphone;
using the GUI to visually select a body part to receive treatment and its treatment protocol from a suggested treatment protocol in order to transmit to a TENS control unit using wireless Bluetooth signals;
transmitting the wireless Bluetooth signals to the TENS control unit from the smartphone;
receiving the wireless Bluetooth signals at a transceiver of the TENS control unit; and
transmitting electrical stimulus pulses from the TENS control unit for application to a user's body during a treatment session in response to receiving the wireless Bluetooth signals.

12. The method of claim 11, further comprising providing electrode pads in electrical communication with the TENS control unit to apply the electrical stimulus pulses to the user's body.

13. (canceled)

14. The method of claim 12, wherein the GUI is installed on the smartphone.

15. The method of claim 12, wherein the GUI is accessible over an intranet or Internet using the smartphone.

16. The method of claim 12, further comprising modulating pulse width, frequency, intensity, or any combination thereof, of the electrical stimulus pulses.

17. The method of claim 16, further comprising selecting a duration of the electrical stimulus pulses.

18. The method of claim 17, further comprising transmitting wireless Bluetooth response signals to the smartphone in response to receiving biofeedback from the user's body.

19. The method of claim 18, further comprising automatically adjusting the control commands to transmit to the TENS control unit in response to receiving the response signals of the biofeedback.

20. A system to remotely control a transcutaneous electrical nerve stimulation (TENS) device, the system comprising:
a TENS control unit;
a transceiver of the TENS control unit configured to receive wireless Bluetooth signals transmitted from a smartphone, and to transmit wireless Bluetooth response signals to the smartphone in response to biofeedback from a user's body;
a controller of the TENS control unit configured to transmit electrical stimulus pulses during a treatment session in response to receiving the wireless Bluetooth signals, wherein the controller is configured to modulate pulse width, frequency, intensity, or any combination thereof, of the electrical stimulus pulses;
electrode pads in electrical communication with the TENS control unit and configured to apply the electrical stimulus pulses to a user's body; and
a graphical user interface accessible using the smartphone and configured for a user to visually select a body part to receive treatment and its treatment protocol from a suggested treatment protocol to transmit to the TENS control unit using the wireless Bluetooth signals.
